(19) United States
(12) Patent Application Publication
(10) Pub. No.: US 2004/0018540 A1
(43) Pub. Date: Jan. 29, 2004

(54) Evaluation method of interferon beta treatment against multiple sclerosis

(75) Inventors: Takashi Yamamura, Kokubunji (JP); Jun-ichi Satoh, Kodaira (JP); Toshiro Saito, Hatoyama (JP); Hiroyuki Tomita, Kawasaki (JP); Masatoshi Narahara, Sayama (JP); Hirokazu Kato, Hatoyama (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/607,050

(22) Filed: Jun. 27, 2003

(30) Foreign Application Priority Data
Jun. 28, 2002 (JP) 2002-188932

(51) Int. Cl. C12Q 1/68; G06F 19/00; G01N 33/48; G01N 33/50

(52) U.S. Cl. 435/6; 702/20

(57) ABSTRACT

The efficacy of an interferon β treatment is evaluated by using a database on correlation between the efficacy of the interferon β treatment and the gene expression levels of at least one interferon induced protein gene, at least one interferon regulation factor gene, and at least one chemokine gene, and determining the expression levels of the genes in messenger RNAs derived from leukocytes in the peripheral blood of a subject.

### FIELD OF THE INVENTION

The present invention relates to an evaluation method of an interferon β treatment against multiple sclerosis.

### BACKGROUND OF THE INVENTION

Multiple sclerosis (hereinafter briefly referred to as MS) is a disease in which fatty sheaths known as “myelin” covering nerve fibers in the brain and spinal cord undergo inflammation, and thereby nervous information is not satisfactorily communicated, thus causing various symptoms such as visual disturbance, dyskinesia, hyposensitivity, and equilibration disorder. The cause of MS has not yet been clarified, and MS is one of chronic diseases which the present medicine cannot cure completely. It is believed to be an autoimmune disease in which the immune system of an individual attacks oneself in error, but the detailed mechanism of its onset has not yet been clarified. It is reported that there are about one million patients with MS in the world.

One of features of MS is that most of patients with MS repeat relapse a number of times. MS is roughly classified as relapsing-remitting MS and progressive MS. In the relapsing-remitting MS, the patients undergo relatively satisfactory recovery when they have undergone an acute phase and enter a remission phase, while the magnitude and duration of the relapse vary from patient to patient. Some of the patients with relapsing-remitting MS undergo increasing aftereffects and progression with an increasing time of relapse. In contrast, in the progressive MS, the patients undergo gradual progression of the disease without significant recovery.

Effects to suppress the relapse of MS have been found in a genetically recombinant interferon β, and this substance has been considered as an effective treatment to suppress the relapse and/or progression of MS. The interferon β includes interferon β-1a commercially available, for example, under the trade name of ABONEX (from Biogen) and interferon β 1b commercially available, for example, under the trade name of BETAFERON (from SCHERING AG). These agents, however, invite flulike symptoms, injection-site reactions, headache, fatigue, depression, and psoriasis as adverse drug reactions. In addition, the efficacy of these agents is found in only about 20% to 30% of patients treated with the agents and is not found in the other patients. Specifically, about 70% to 80% of patients treated with the interferon β suffer adverse drug reactions alone without the effects of reducing the frequency of relapse and retarding of the progression of physical disorders. Strong demands have therefore been made to develop an evaluation method of the efficacy of the treatment at early stages in the treatment to thereby reduce the number of patients suffering from such adverse drug reactions. Conventional evaluation methods of the efficacy include, for example, magnetic resonance imaging (MRI) tests, evoked potential tests, and spinal tap. The MRI tests can differentiate active foci from cured foci by using gadolinium as a contrast medium and are very useful, but cannot detect every focus. The evoked potential tests determine the presence or absence of a focus on the neurotransmission pathway by applying visual, somatic and/or auditory stimuli to a subject, and determining the speed and intensity of signals transmitting on the neurotransmission pathway. The spinal tap detects the presence or absence of a focus by sampling a cerebrospinal fluid flowing around the brain and spinal cord and determining the amounts of leukocytes, antibodies (immunoglobulin G; TgG) and myelin basic proteins in the spinal fluid and is very useful. However, this test requires puncture on the back of the subjects and puts an enormous load or burden on subjects. These conventional evaluation methods cannot significantly easily evaluate the efficacy of the interferon β treatment at early stages with a high detection sensitivity and less burden on the subjects.

### SUMMARY OF THE INVENTION

Accordingly, an object of the present invention is to provide an evaluation method that can easily and reliably evaluate the efficacy of an interferon β treatment on a patient with MS with less burden on the patient.

After intensive investigations to achieve the above objects, the present inventors have found that the efficacy of an interferon β treatment can be evaluated by determining the expression levels of a specific gene cluster in leukocytes derived from the peripheral blood of a subject according to an easy procedure such as DNA chips. The present invention has been accomplished based on these findings.

The present invention will be illustrated in more detail below.

Specifically, the present invention provides, in an aspect, an evaluation method of an interferon β treatment, including quantifying each kind of messenger RNA molecule derived from peripheral blood leukocytes of a subject to thereby determine the expression levels of at least one interferon induced protein gene, at least one interferon regulation factor gene, and at least one chemokine gene; and evaluating the efficacy of the interferon β treatment on the subject-based on the measured gene expression levels and a database including data on correlation between the efficacy of the interferon β treatment and the expression levels of the at least one interferon induced protein gene, the at least one interferon regulation factor gene, and the at least one chemokine gene.

In the evaluation method, the at least one interferon induced protein gene may be at least one gene having a symbol name selected from the group consisting of IFIT1, IFIT4, G1P3, and ISG15, the at least one interferon regulation factor gene may be at least one gene having a symbol name selected from the group consisting of IRF1, IRF2, IRF3, IRF4, IRF5, IRF6, and IRF7, and the at least one chemokine gene may be at least one gene having a symbol name selected from the group consisting of SCYA2, SCYA22, SCYA5, SCYB14, CCR5, CXCR3, CCR4, CCR3, CCR8, CXCR5, MIP-1α, MIG, IP-10, TARC, MDC, and SDF-1.

The evaluation method preferably further includes using probes corresponding to at least one interleukin gene having a symbol name selected from the group consisting of IL4, IL10, IL12A, IL12B, and IL18, and at least one transforming growth factor gene having a symbol name selected from the group consisting of TGFA, TGFB1, TGFB2, and TGFB3 to thereby evaluate the efficacy based on the database and the expression levels of the aforementioned genes in addition to those of the interferon induced protein gene, the interferon regulation factor gene and the chemokine gene.

MS is supposed to be an autoimmune disease caused by malfunctions of the-immune system. The interferon β is believed to repair abnormality or disorder of the immune system. It has been found that the interferon β improves the functions of suppressor T cells, suppresses the production of some of cytokines, i.e., lymphotoxin, tumor necrosis factor (TNF), and interferon gamma (INFγ) and accelerates the production of transforming growth factor beta (TGFβ). The patients with MS exhibit decreased functions of the suppressor T cells, a kind of lymphocytes. However, it may be very risky to determine or evaluate the abnormalities and repair thereof of the immune system by observing individual behaviors of the suppressor T cells, lymphotoxin, TNF, INFγ, and TGFβ. This is because the immune system is a very complicated system serving as an intracellular transmission network of signals among plural types of cells including T cells and B cells. The present inventors have aimed at the development of a method for evaluating the immune system by observing the behavior of a gene cluster in a broader range.

A technology for determining gene expression in a sample cell using a DNA array or DNA chip has received attention. In this technology, a DNA array or DNA chip is prepared by immobilizing a multitude of DNA fragments having different sequences to different positions of a substrate. Messenger RNAs are extracted from a target cell in which the gene expression is to be determined, and fluorescence-labeled or radio-isotope-labeled reverse transcripts of the messenger RNAs are placed on the DNA array or DNA chip for hybridization. The levels of hybridization of the reverse transcripts on the different positions of the DNA fragments having different sequences are determined respectively to thereby determine the gene expression in the sample cell. The present inventors have made an exhaustive study of a gene cluster that exhibits varied gene expression as a result of the interferon β treatment by using the DNA array technique.

As a sample, leukocytes contributing to the immune system were collected from the peripheral blood of subjects. The use of samples collected from the peripheral blood can significantly mitigate the burden on the subjects. The study was made on a group of ten patients diagnosed as relapsing-remitting MS in comprehensive consideration of the MRI tests, evoked potential tests, spinal tap, and clinical findings. The present inventors have made an exhaustive study of a gene cluster that exhibits varied gene expression before and after interferon β treatment by using the DNA array technique. A DNA chip (drug response DNA chip, available from Hitachi, Ltd.) comprising about 1260 human genes being immobilized thereon was used. These genes relate to, for example, cytokines, signal transduction, growth factors, oncogenes, and apoptosis. The blood was drawn from the patient group three times, i.e., before treatment, three months into the treatment and six months into the treatment. In contrast, a reference (control) sample was prepared in the following manner. The peripheral blood was collected from three healthy volunteers in the same manner as in the patient group. RNA samples were extracted from leukocytes in their peripheral blood, and the three samples from the three healthy subjects were mixed, the resulting mixture was subjected to an RNA amplification reaction using in vitro transcription. The amplified RNA was used as a common reference sample among all the patient samples.

The total RNA was extracted from the leukocytes derived from the peripheral blood of the patient group using a TRIzol reagent (trade name, available from Invitrogen Corp., Carlsbad, Calif., USA). Using the extracted total RNA, cDNA labeled with Cy5 was prepared by a reverse transcription reaction using Cy5-dCTP. Separately, from the reference sample derived from the healthy subjects, cDNA labeled with Cy3 was prepared by a reverse transcription reaction using Cy3-dCTP. These cDNAs were mixed in equal proportions, and the mixture was placed on the DNA chip to perform hybridization at 62° C. for 12 hours. After rinsing, fluorescence intensities of individual spots were determined using a scanner (available from GSI-Lumonics Inc. under the trade name of ScanArray 5000). The ratios of expression levels of the sample derived from the patient to those of the reference sample were determined. The comparative test in gene expression using the entire DNA chip can easily determine changes or variations in expression levels among patients or changes with sampling time in the same patient, since the ratios of expression levels with respect to the common reference sample are determined.

The data were analyzed in the following manner. A gene cluster that exhibited varied expression levels after the initiation of the interferon β treatment (three months later and six months later) as compared with those before the initiation of the treatment was extracted in each patient group. Likewise, a gene cluster that exhibited varied expression levels three months after the initiation of the treatment as compared with those six months after the initiation of the treatment. To extract these gene clusters, two groups each including ten samples with different time series were subjected to t-test, and gene clusters which exhibited statistically significant varied expression levels between the two groups even in consideration of differences among individuals (among samples) were selected. A Bayesian t-test reported by A. Long et al. in Journal of Biological Chemistry 276, 19937-19944 (2001) was used as the t-test at a permitted false positive rate of 0.25. The results are shown in Table 1-1,1-2 and 1-3. Gene clusters that exhibited statistically significant differences in expression levels between the two groups were selected as varied gene clusters. The selected varied gene clusters are shown in Table 2. Genes relating to interferon induced proteins, interferon regulation-factors, and chemokines are selected as the variant gene clusters. These gene clusters are assessed to be markedly affected by the interferon β to thereby vary their expression.

TABLE 1-1
Differentially expressed genes selected by t-test between before and
after interferon beta treatment and their fold change compared with
control sample

| | Before interferon beta treatment | | | | | | | | | |
|---|---|---|---|---|---|---|---|---|---|---|
| Name | No.1 | No.2 | No.3 | No.4 | No.5 | No.6 | No.7 | No.8 | No.9 | No.10 |
| ISI15 | 0.41 | 1.15 | 1.21 | 0.48 | 0.81 | 0.69 | 1.52 | 1.44 | 1.30 | 0.93 |
| IFI27 | 1.45 | 0.81 | 2.05 | 2.47 | 1.86 | 1.43 | 1.23 | 1.41 | 2.53 | 1.16 |
| IFIT1 | 0.46 | 1.33 | 0.25 | 0.43 | 0.28 | 0.67 | 0.52 | 0.26 | 0.30 | 1.00 |
| RPC39 | 0.34 | 0.37 | 0.94 | 0.40 | 1.05 | 0.33 | 1.74 | 1.34 | 4.78 | 1.40 |
| GIP3 | 0.17 | 0.98 | 0.95 | 0.26 | 1.24 | 0.89 | 0.61 | 0.46 | 0.64 | 0.63 |
| IRF7 | 0.06 | 1.16 | 0.34 | 0.05 | 0.34 | 0.19 | 0.27 | 0.37 | 0.16 | 0.44 |
| SULTIC1 | 0.26 | 0.23 | 0.48 | 0.30 | 0.56 | 1.06 | 0.45 | 0.28 | 0.25 | 0.56 |
| IFIT4 | 2.65 | 1.10 | 0.77 | 1.49 | 0.91 | 1.99 | 0.93 | 0.92 | 0.76 | 1.05 |
| SCYA5 | 5.72 | 7.79 | 10.18 | 2.25 | 8.19 | 1.04 | 8.12 | 6.67 | 2.14 | 2.38 |
| SCYB14 | 3.44 | 1.98 | 7.74 | 4.56 | 14.49 | 32.39 | 4.83 | 3.35 | 5.32 | 2.66 |
| SCYA2 | 5.86 | 3.68 | 3.24 | 4.89 | 2.11 | 3.05 | 3.72 | 2.29 | 2.13 | 1.35 |
| TSC22 | 0.05 | 0.11 | 0.64 | 0.05 | 0.46 | 0.15 | 3.38 | 1.38 | 1.78 | 0.27 |
| ABCB2 | 0.07 | 0.10 | 0.25 | 0.07 | 0.24 | 0.20 | 0.20 | 0.24 | 0.26 | 0.67 |
| TAF2B | 1.31 | 1.43 | 13.90 | 5.04 | 16.27 | 32.08 | 5.00 | 7.77 | 10.54 | 0.70 |
| GADD45G | 0.42 | 1.05 | 0.83 | 0.60 | 1.15 | 0.77 | 0.69 | 0.97 | 1.07 | 0.54 |
| TNFAIP6 | 0.82 | 1.63 | 0.43 | 1.14 | 1.34 | 1.52 | 0.78 | 1.05 | 0.71 | 0.46 |
| SP100 | 0.04 | 0.08 | 0.61 | 0.06 | 0.37 | 0.18 | 0.18 | 0.37 | 0.16 | 0.87 |
| IFITM1 | 0.87 | 0.45 | 0.51 | 0.90 | 0.80 | 1.42 | 1.17 | 0.91 | 0.86 | 1.26 |
| RAB11A | 0.09 | 0.11 | 0.53 | 0.15 | 0.49 | 0.22 | 1.78 | 1.08 | 3.04 | 0.55 |
| CCND3 | 0.11 | 0.13 | 0.45 | 0.07 | 0.58 | 0.14 | 1.31 | 2.55 | 1.96 | 0.67 |
| PRKCABP | NA | 1.54 | 1.98 | NA | 0.79 | NA | 1.80 | NA | NA | NA |
| TPST2 | 0.08 | 0.14 | 0.79 | 0.16 | 0.87 | 0.20 | 1.68 | 2.14 | 4.37 | 0.61 |
| CRHR1 | 3.26 | 1.53 | 3.02 | 1.71 | 2.28 | 2.08 | 3.19 | 1.66 | 3.59 | 3.32 |
| GNA13 | NA | 3.38 | 3.03 | NA | 0.34 | NA | 64.94 | 0.46 | NA | NA |
| AKAP4 | NA | 3.65 | 1.80 | 3.50 | 5.02 | 3.27 | NA | 1.98 | 4.10 | 1.01 |
| SLC7A1 | NA | 6.24 | 1.94 | NA | 5.00 | 1.06 | 64.26 | 1.53 | NA | 2.74 |
| TLR5 | 0.31 | 0.40 | 0.78 | 0.40 | 0.43 | 0.40 | 0.50 | 0.36 | 0.37 | 0.54 |

| | Three months after interferon beta treatment | | | | | | | | | | |
|---|---|---|---|---|---|---|---|---|---|---|---|
| Name | No.1 | No.2 | No.3 | No.4 | No.5 | No.6 | No.7 | No.8 | No.9 | No.10 | p-value |
| ISG15 | 2.52 | 3.20 | 31.49 | 2.99 | 10.76 | 4.16 | 2.44 | 5.47 | 4.53 | 15.90 | 3.8E−15 |
| IFI27 | 0.99 | 3.57 | 31.67 | 3.70 | 3.63 | 6.35 | 6.74 | 3.13 | 7.24 | 15.83 | 1.6E−12 |
| IFIT1 | 1.17 | 2.05 | 5.28 | 0.64 | 6.36 | 2.90 | 1.44 | 3.73 | 6.21 | 18.80 | 5.1E−09 |
| RPC39 | 0.85 | 11.48 | 4.14 | 0.48 | 4.81 | 10.38 | 3.73 | 5.58 | 11.82 | 0.94 | 1.7E−08 |
| GIP3 | 1.58 | 2.56 | 5.08 | 0.30 | 9.75 | 5.22 | 0.80 | 4.01 | 4.70 | 5.30 | 5.3E−07 |
| IRF7 | 1.35 | 0.41 | 2.46 | 0.17 | 2.53 | 1.06 | 0.30 | 1.79 | 0.49 | 6.43 | 8.2E−07 |
| SULT1C1 | 0.43 | 0.92 | 2.03 | 0.22 | 2.31 | 1.26 | 0.46 | 1.78 | 0.65 | 2.25 | 4.5E−05 |
| IFIT4 | 2.01 | 1.11 | 4.55 | 1.04 | 2.83 | 1.54 | 1.96 | 1.42 | 1.98 | 28.74 | 5.7E−05 |
| SCYA5 | 2.29 | 0.78 | 9.89 | 1.15 | 0.81 | 0.95 | 1.21 | 2.53 | 2.29 | 1.81 | 8.7E−05 |
| SCYB14 | 2.04 | 3.08 | 4.94 | 7.08 | 1.99 | 6.51 | 3.89 | 1.53 | NA | 2.23 | 9.9E−05 |
| SCYA2 | 2.52 | 1.93 | 18.95 | 4.30 | 1.80 | 9.30 | 11.98 | 1.67 | 7.42 | 7.84 | 1.3E−04 |
| TSC22 | 1.04 | 2.92 | 3.19 | 0.14 | 1.00 | 9.34 | 1.51 | 4.97 | 6.84 | 0.68 | 1.3E−04 |
| ABCB2 | 0.59 | 0.36 | 0.80 | 0.07 | 1.08 | 0.81 | 0.24 | 1.01 | 0.50 | 1.11 | 1.6E−04 |
| TAF2B | 1.18 | 6.13 | 9.19 | 2.17 | 6.17 | 8.55 | 6.36 | 4.49 | 6.52 | 0.88 | 1.7E−04 |
| GADD45G | 0.58 | 1.78 | 2.88 | 0.79 | 2.45 | 3.41 | 1.65 | 4.02 | 3.46 | 0.65 | 1.9E−04 |
| TNFAIP6 | 1.88 | 2.11 | 16.09 | 0.93 | 3.09 | 1.21 | 0.50 | 3.49 | 2.36 | 0.77 | 5.9E−04 |
| SP100 | 0.69 | 0.36 | 1.06 | 0.05 | 0.98 | 0.88 | 0.19 | 1.01 | 0.50 | 1.29 | 6.1E−04 |
| IFITM1 | 0.99 | 0.75 | 2.42 | 1.07 | 4.10 | 2.40 | 1.70 | 2.37 | 1.88 | 5.28 | 6.5E−04 |
| RAB11A | 0.47 | 2.42 | 2.20 | 0.13 | 2.52 | 3.36 | 1.86 | 5.33 | 6.04 | 0.60 | 8.7E−04 |
| CCND3 | 0.39 | 2.33 | 0.80 | 0.11 | 6.11 | 2.69 | 0.56 | 3.08 | 0.49 | 1.33 | 1.3E−03 |
| PRKCABP | NA | 0.54 | 0.97 | NA | NA | NA | 0.31 | NA | NA | 0.15 | 1.8E−03 |
| TPST2 | 0.59 | 2.80 | 1.61 | 0.28 | 4.54 | 2.11 | 0.73 | 4.68 | 2.01 | 0.48 | 1.1E−02 |
| CHRHR1 | 1.42 | 0.95 | NA | 4.04 | 1.30 | 0.93 | 2.56 | 1.19 | 0.20 | 1.01 | 1.6E−02 |
| GNA13 | 2.97 | 2.51 | NA | NA | 1.50 | 1.61 | NA | NA | 2.95 | 1.54 | 3.0E−08 |
| AKAP4 | 0.97 | 2.80 | NA | 3.05 | 1.16 | 2.08 | NA | 1.18 | 1.59 | 1.44 | 3.2E−06 |
| SLC7A1 | 2.78 | 3.57 | NA | 7.80 | 2.10 | 2.34 | NA | NA | 4.77 | 1.96 | 2.3E−04 |
| TLR5 | 0.40 | 2.49 | 1.69 | 1.13 | 1.14 | 1.35 | 1.52 | 1.20 | 0.50 | 0.56 | 3.0E−04 |

*The balue presents fold change compared with control sample.
*NA: not available

TABLE 1-2
Differentially expressed genes selected by t-test
between three months and six months after interferon
beta treatment and their change compared with control
sample

| | Three months after interferon beta treatment | | | | | | | | | |
|---|---|---|---|---|---|---|---|---|---|---|
| Name | No.1 | No.2 | No.3 | No.4 | No.5 | No.6 | No.7 | No.8 | No.9 | No.10 |
| ISG15 | 2.52 | 3.20 | 31.49 | 2.99 | 10.76 | 4.16 | 2.44 | 5.47 | 4.53 | 15.90 |
| IFIT4 | 5.04 | 2.01 | 16.60 | 3.92 | 6.55 | 2.42 | 1.47 | 7.14 | 3.15 | 7.98 |
| SCYA22 | 1.26 | 1.81 | 17.48 | 4.86 | 1.17 | 1.895 | NA | 37.04 | 2.88 | 1.51 |
| ARHGEF1 | 3.05 | 0.75 | 3.50 | 3.75 | 1.38 | 0.84 | 0.47 | 8.91 | 1.34 | 0.90 |

| | Six months after interferon beta treatment | | | | | | | | | | |
|---|---|---|---|---|---|---|---|---|---|---|---|
| Name | No.1 | No.2 | No.3 | No.4 | No.5 | No.6 | No.7 | No.8 | No.9 | No.10 | p [symbol] |
| ISG15 | 0.72 | 1.57 | 2.37 | 3.78 | 6.54 | 3.32 | 2.66 | 0.85 | 4.19 | 1.10 | 1.9E−06 |
| IFIT4 | 4.06 | 0.97 | 2.42 | 1.76 | 2.27 | 4.50 | 3.75 | 4.59 | 29.95 | 99.25 | 4.6E−05 |
| SCYA22 | NA | 1.13 | 2.79 | 1.55 | 4.97 | 1.51 | 2.31 | 1.79 | 1.12 | 1.27 | 4.7E−05 |
| ARHGEF1 | 22.31 | 1.54 | 4.30 | 1.74 | 1.65 | 7.36 | 8.72 | 1.79 | 3.92 | 12.74 | 5.2E−05 |

The value presents fold change compared with control sample.
*NA: not available

TABLE 1-3
Differentially expressed genes selected by t-test
between before interferon beta treatment and six
months after the treatment and their fold change
compared with control sample

| | Before interferon beta treatment | | | | | | | | | |
|---|---|---|---|---|---|---|---|---|---|---|
| Name | No.1 | No.2 | No.3 | No.4 | No.5 | No.6 | No.7 | No.8 | No.9 | No.10 |
| COX7A1 | NA | 6.63 | NA | NA | NA | NA | NA | NA | NA | NA |
| GNA13 | NA | 3.38 | 3.03 | NA | 0.34 | NA | 64.94 | 0.46 | NA | NA |
| IFIT4 | 1.85 | 2.47 | 1.76 | 1.35 | 1.14 | 1.64 | 2.48 | 2.22 | 1.61 | 1.72 |
| MIG | 19.63 | 21.30 | 3.14 | 3.68 | 9.36 | 32.52 | 8.44 | 0.93 | NA | 2.78 |
| AKAP4 | NA | 3.65 | 1.80 | 3.50 | 5.02 | 3.27 | NA | 1.98 | 4.10 | 1.01 |
| SLC7A1 | NA | 6.24 | 1.94 | NA | 5.00 | 1.06 | 64.26 | 1.53 | NA | 2.74 |
| ARHGEF1 | 5.12 | 0.86 | 2.27 | 2.67 | 1.29 | 1.81 | 0.75 | 2.62 | 2.04 | 4.14 |
| IRF7 | 0.28 | NA | 0.52 | 0.97 | 0.24 | 0.23 | 0.10 | 0.27 | 0.31 | 0.47 |
| GIP3 | 0.34 | 0.07 | 0.78 | 0.51 | 0.57 | 0.47 | 0.29 | 0.30 | 0.45 | 0.48 |
| IFIT1 | 1.11 | 1.07 | 1.20 | 0.92 | 0.63 | 0.95 | 2.56 | 1.25 | 0.39 | 1.08 |
| TLR5 | 0.31 | 0.40 | 0.78 | 0.40 | 0.43 | 0.40 | 0.50 | 0.36 | 0.37 | 0.54 |
| GHSR | 14.89 | 4.77 | 2.48 | 4.74 | 5.03 | 6.21 | 47.90 | 2.38 | 5.97 | 4.70 |
| CCNB2 | 0.81 | 0.15 | 0.34 | 1.21 | 1.31 | 2.59 | 0.36 | 0.56 | 0.51 | 1.24 |
| GHRHR | NA | 4.22 | NA | 1.71 | 3.66 | NA | NA | NA | 2.28 | NA |
| PTPRC | 0.20 | 0.01 | 0.26 | 0.52 | 0.02 | 0.09 | 0.06 | 0.41 | 0.07 | 0.75 |

| | Six months after interferon beta treatment | | | | | | | | | | |
|---|---|---|---|---|---|---|---|---|---|---|---|
| Name | No.1 | No.2 | No.3 | No.4 | No.5 | No.6 | No.7 | No.8 | No.9 | No.10 | p [symbol] |
| COX7A1 | NA | 0.37 | 1.86 | NA | NA | 2.33 | 2.41 | 3.26 | 0.84 | NA | 0.00E+00 |
| GNA13 | NA | 0.69 | NA | 2.37 | 3.87 | 0.97 | 2.34 | 1.97 | 0.81 | 1.44 | 2.07E−11 |
| IFIT4 | 4.06 | 0.97 | 2.42 | 1.76 | 2.27 | 4.50 | 3.75 | 4.59 | 29.95 | 99.25 | 7.86E−10 |
| MIG | 2.66 | 1.60 | 1.98 | 4.57 | 9.42 | 10.33 | 2.48 | 3.35 | 1.39 | 1.50 | 2.77E−07 |
| AKAP4 | NA | 0.92 | 1.06 | 2.06 | 7.46 | 0.73 | 1.13 | 2.37 | 1.14 | 1.28 | 1.46E−06 |
| SLC7A1 | NA | 1.09 | 2.51 | 2.57 | 11.71 | 1.30 | 1.85 | 2.88 | 1.13 | 1.30 | 2.28E−06 |
| ARHGEF1 | 22.31 | 1.54 | 4.30 | 1.74 | 1.65 | 7.36 | 8.72 | 1.79 | 3.92 | 12.74 | 4.17E−06 |
| IRF7 | 0.84 | NA | 0.66 | 0.57 | 0.46 | 1.43 | 1.34 | 0.97 | 2.05 | 4.45 | 9.08E−06 |
| GIP3 | 0.42 | 2.02 | 1.09 | 1.13 | 0.54 | 1.22 | 1.53 | 0.82 | 3.08 | 4.63 | 2.47E−05 |
| IFIT1 | 2.27 | 0.56 | 1.51 | 0.81 | 1.30 | 1.17 | 1.73 | 2.74 | 6.96 | 28.62 | 4.84E−05 |
| TLR5 | 0.48 | 3.52 | 3.16 | 0.56 | 0.39 | 0.34 | 0.40 | 0.25 | 1.74 | 1.83 | 5.06E−05 |
| GHSR | NA | 0.72 | 2.30 | 3.28 | 12.79 | 4.26 | 1.83 | 3.63 | 1.42 | 2.87 | 1.04E−04 |
| CCNB2 | 2.08 | 4.88 | 0.95 | 0.99 | 0.75 | 5.24 | 1.35 | 1.14 | 1.26 | 1.50 | 1.21E−02 |
| GHRHR | NA | 0.43 | 0.73 | 0.69 | 1.85 | 1.95 | 0.97 | NA | 0.51 | 0.80 | 1.52E−02 |
| PTPRC | 0.90 | 0.59 | 0.35 | 0.17 | 0.18 | 0.36 | 0.94 | 0.21 | 0.62 | 0.50 | 1.77E−02 |

*The value presents fold change compared with control sample.
*NA: not available

TABLE 2

| Seq. | | | | GenBank |
|---|---|---|---|---|
| No. | Symbol | Name | Category | (Acc. No.) |
| 1 | RAB11A | Homo sapiens rab11a GTPase mRNA, complete cds. | oncogene | AF000231 |
| 2 | CCNB2 | Human cyclin B2 mRNA, complete cds | CellCycle | AF002822 |
| 3 | TAF2B | TATA box binding protein (TBP)-associated factor, RNA | polymerase, | AF040701 |
| | | polymerase II, B, 150 kD | TF | |
| 4 | TPST2 | Homo sapiens tyrosylprotein sulfotransferase-2 mRNA | sulfotransfera | AF049891 |
| 5 | SCYB14 | Homo sapiens CXC xhemokine BRAK mRNA, Small inducible | Cytokine | AF073957 |
| | | cytokine subfamily B (Cys-X-Cys), member 14 | | |
| 6 | IFIT4 | Homo sapiens interferon induced tetratricopeptide protein IFI60 | Cytokine | AF083470 |
| | | (IFIT4) mRNA, complete cds | | |
| 7 | PRKCAB | Novel human mRNA similar to mouse gene PICK1; Protein | Signal | AL049654 |
| | P | kinase C, alpha binding protein | | |
| 8 | IFITM1 | Human interferon-inducible protein 9-27 mRNA, complete cds | Cytokine | J04164 |
| 9 | GNA13 | Human guanine nucleotide regulatory protein (G13) mRNA; | Signal | L22075 |
| | | Guanine nucleotide binding protein (G protein), alpha 13 | | |
| 10 | CRHR1 | Human corticotropin releasing factor receptor mRNA | corticotrophin | L23332 |
| | | | (ACTH) | |
| 11 | ISG15 | Human interferon-induced 17-kDa/15-kDa protein mRNA | Cytokine | M13755 |
| | | (interferon-stimulated protein, 15 kDa) | | |
| 12 | SCYA5 | Human T cell-specific protein (RANTES) mRNA, Small | Cytokine | M21121 |
| | | inducible cytokine A5 | | |
| 13 | TNFAIP6 | Tumor necorsis factor, alpha-induced protein 6 | Cytokine, | M31165 |
| | | | Signal | |
| 14 | SP100 | Human nuclear autoantigen (SP-100) mRNA | Signal | M60618 |
| 15 | CCND3 | Homo sapiens cyclin D3 (CCND3) mRNA, complete cds | CellCycle | M92287 |
| 16 | COX7A1 | Homo sapiens cytochrome c oxidase subunit VIIa polypeptide | mitochondria & | NM_001864 |
| | | 1(muscle) (COX7A1), nuclear gene encoding mitochondrial | stress | |
| 17 | SLC7A1 | Homo sapiens solute carrier family 7 (cationic amino acid | hyperosmotic | NM_003045 |
| | | transporter, y+ system), member 1 | stress | |
| 18 | GHSR | Homo sapiens growth hormone secretagigue receptor (GHSR) | GH | NM_004122 |
| 19 | GADD45 | Homo sapiens growth arrest and DNA-damage-inducible, | DNA-damage- | NM_006705 |
| | G | gamma (GADD45G) | inducible | |
| 20 | GHRHR | Homo sapiens growth hormone releasing hormone receptor | GH | NM_000823 |
| 21 | SCYA2 | monocyte chemoattractant protein-I [human, mRNA, 739 nt], | Cytokine, | S71513 |
| | | MCP-I | | |
| 22 | TSC22 | Human putative regulatory protein TGF-beta-stimulated clone | GF | U35048 |
| | | 22 homolog (TSC22) | | |
| 23 | IRF7 | Homo sapiens interferon regulatory factor 7A mRNA, complete | Cytokine | U53830 |
| | | cds | | |
| 24 | ARHGEF1 | Human guanine nucleotide exchange factor p115-RhoGEF | Signal | U64105 |
| | | mRNA, partial cds; Rho guanine nucleotide exchange factor | | |
| 25 | SULTC1 | Human sulfotransferases mRNA family 1C, member 1 (SULT1C1) | sulfotransfera | U66036 |
| 26 | SCYA22 | Human macrophage-derived chemokine precursor (MDC) | Cytokine | U83171 |
| | | mRNA; Small inducible cytokine subfamily A (Cys-Cys). | | |
| 27 | TLR5 | Homo sapiens Toll-like receptor 5 (TLR5) mRNA, partial cds. | Signal | U88881 |
| 28 | RPC39 | polymerase (RNA) III (DNA directed) (39 kD) | polymerase | U93869 |
| 29 | G1P3 | Human interferon-inducible mRNA fragment (cDNA 6-16). | Cytokine | X02492 |
| 30 | IFIT1 | Human mRNA for 56-KDa protein induced by interferon | Cytokine | X03557 |
| 31 | IFI27 | H. sapiens p27 mRNA (interferon, alpha-inducible protein 27) | Cytokine | X67325 |
| 32 | MIG | H. sapiens Humif mRNA | Cytokine | X72755 |
| 33 | PTPRC | Human mRNA for T200 leukocyte common antigen (CD45, LC- | Signal | Y00062 |

Next, the extracted gene clusters were arrayed in order of expression level in each patient at each sampling time to form a matrix, and a cluster analysis was performed to thereby group the patient group containing ten patients. In the analysis, agglomerative and divisive clustering procedures were used based on unweighted Euclidian distances between clusters. FIGS. 1A and 1B are dendrogram representations of hierarchical clustering according to the agglomerative and divisive clustering procedures, respectively. The heights in these serve as an index of the distance between clusters. FIGS. 1A and 1B show that the patient No. 10 is in another hierarchy than the other nine patients. With reference to clinical data, the patient No. 10 alone shows remarkable clinical therapeutic effects of the interferon β treatment. These results show that patients exhibiting remarkable therapeutic effects can be selected by cluster analysis using gene clusters that exhibit statistically significantly varied expression by the interferon β treatment as a marker.

Next, since MS is believed to be an autoimmune disease, the group of ten patients was subjected to clustering further using, as the marker gene clusters, genes of ligands or receptors of chemokines having symbol names of CCR5, CXCR3, CCR4, CCR3, CCR8, CXCR5, MIP-1α, IP-10, TARC, MDC, and SDF-1, interleukin genes having symbol names of IL4, IL10, IL12A, IL12B, and IL18, and transforming growth factor genes having symbol names of TGFA, TGFB1, TGFB2, and TGFB3. FIGS. 2A and 2B are dendrogram representations of hierarchical clustering according to the agglomerative and divisive clustering procedures, respectively. The result in this clustering shows that the patient No. 10 alone is in another hierarchy than the other nine patients as in the aforementioned clustering. However, the result further shows that more hierarchically clear clustering can be performed according to this technique, since the agglomerative coefficient and divisive coefficient as indices how clearly hierarchically the clustering is performed approach 1 by adding the genes relating to the chemokines, interleukins, and transforming growth factors. As is described above, these results show that the presence or absence of the efficacy of the interferon β treatment can be clearly evaluated by statistically analyzing variation in gene expression levels of a patient group using a specific gene cluster as a marker. The present invention has been accomplished based on these results and findings. FIG. 3 is a schematic diagram illustrating the present invention. According to the present invention, the interferon β treatment is evaluated by drawing the peripheral blood from a subject, extracting RNAs from the peripheral blood, and analyzing the expression profile of the RNAs. FIG. 3 illustrates a procedure using a DNA chip (DNA array) 1. The DNA chip 1 comprises probe DNAs 2 immobilized thereon. The probe DNAs 2 correspond to genes selected in the present invention and-are subjected to hybridization with cDNA labeled with fluorescent dye prepared from the RNAs extracted from the subject. The hybridization is detected using an excitation light source and a fluorescence detector controlled by a controller computer 4. Even a small amount of about 2 ml of the blood drawn can be sufficiently analyzed after an RNA amplification reaction. The procedure of determining the expression levels of genes for use in the present invention is not limited to the procedure using such a DNA chip but also includes, for example, quantitative PCR and Northern blotting.

The data analysis procedure for use in the present invention is also not limited to clustering and includes, for example, algorithms of machine learning such as support vector machines. Regardless of whether the analysis procedure is a supervised leaning algorithm or a non-supervised leaning algorithm, the presence or absence of the efficacy on subjects can be evaluated with reference to a database based on the relation between gene expression data and clinical data, and the database becomes more sufficient by adding data of subjects at any time to the database. Accordingly, the efficacy can be more precisely evaluated. This is one of remarkable features of the evaluation method of the present invention.

Further objects, features and advantages of the present invention will become apparent from the following description of the preferred embodiments with reference to the attached drawings.

### BRIEF DESCRIPTION OF THE DRAWINGS

FIGS. 1A and 1B show analytical results in hierarchical clustering;

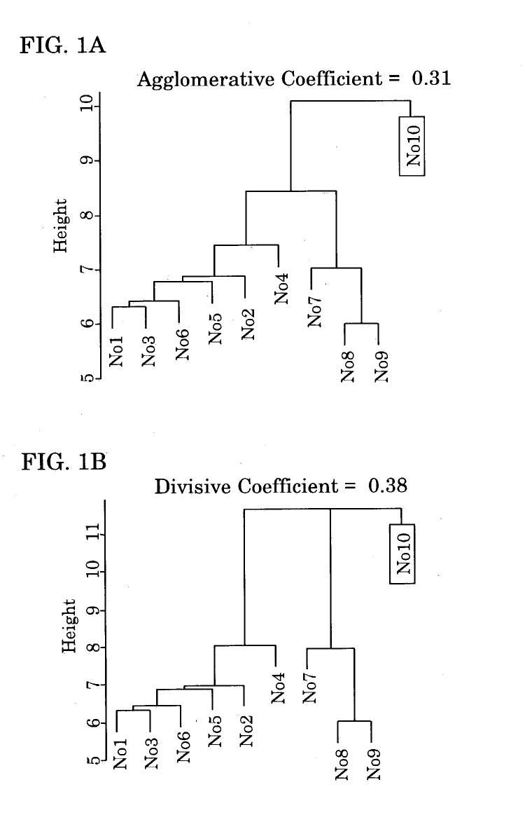

FIGS. 2A and 2B show analytical results in hierarchical clustering;

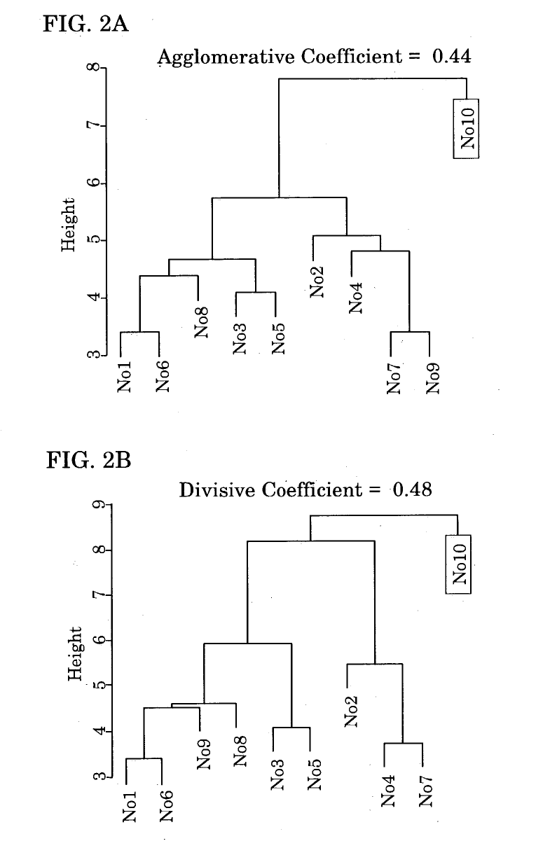

FIG. 3 is a schematic diagram illustrating the present invention; and

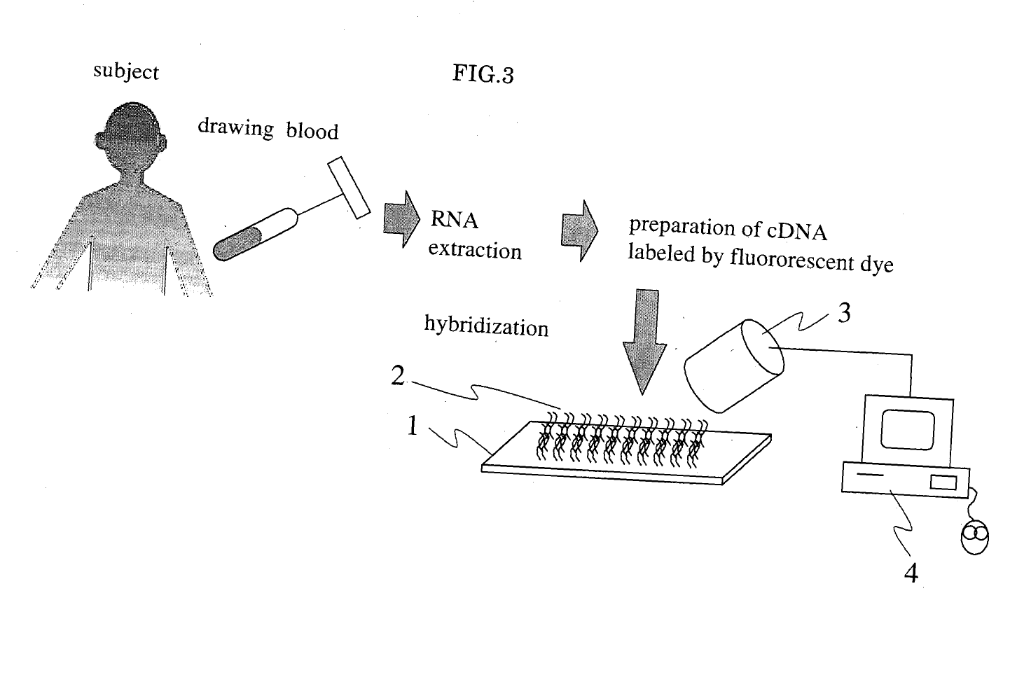

FIG. 4 shows analytical results in hierarchical clustering.

### DESCRIPTION OF THE PREFERRED EMBODIMENTS

The present invention will be illustrated in further detail with reference to an example below, which is not intended to limit the scope of the present invention.

### EXAMPLE 1

In the present example, the efficacy of an interferon β treatment on subjects was evaluated by analyzing gene expression levels in the subjects, and further analyzing the results with reference to a database including data of a patient group in which the presence or absence of the efficacy had been clinically clarified.

In the database, the data of the ten patients mentioned above were used. The efficacy on five new subjects who had been treated with-the interferon β was evaluated. The five subjects were patients synthetically diagnosed as relapsing-remitting MS comprehensively based on the results of MRT tests, evoked potential tests, spinal tap and clinical findings. At the time when the blood was drawn before treatment and three months after the initiation of the treatment, they were in remission with relatively mitigated symptoms. Each 2 milliliters of the peripheral blood was drawn from each subject using a PAXgene Blood RNA System (available from QIAGEN K.K.), and total RNA was extracted from the peripheral blood in a yield of 5 to 10 micrograms.

Next, 5 micrograms of the total RNA was subjected to annealing with an oligo(dT) 24 primer having a T7 promoter sequence, and a first strand DNA was synthesized.

Next, a second strand DNA having the T7 promoter sequence was synthesized using the first strand DNA as a template. An RNA was synthesized using a T7 RNA polymerase and the second strand DNA as a template.

Next, 6 micrograms of the amplified RNA was subjected to annealing with a random hexamer and to a reverse transcription reaction to incorporate Cy5-dCTP in its strand to thereby yield a fluorescence-labeled cDNA.

A control sample was prepared in the following manner. Each 4 milliliters of the peripheral blood was drawn from each of three healthy volunteer subjects using a PAXgene Blood RNA System (available from QIAGEN K.K.), and total RNA was extracted from the peripheral blood. Each 10 micrograms of the total RNAs of the three subjects were mixed, the mixture was subjected to the RNA amplification reaction and reverse transcription reaction and thereby yielded fluorescence-labeled cDNA as a common control sample.

The Cy5-cDNA prepared from each patient sample and the Cy3-cDNA as the common control sample were mixed in equal proportions of 4 micrograms each, and the mixture was placed on the DNA chip (drug response DNA chip, available from Hitachi, Ltd.) for hybridization at 62° C. for 12 hours. After rinsing, fluorescence intensities of individual spots were determined using a scanner (available from GSI Lumonics Inc. under the trade name of ScanArray 5000). The ratios of expression intensities in individual genes between the control sample and each of the patient samples were determined using digitizing software. (available from GSI Lumonics Inc, under the trade name of QuantAssay).

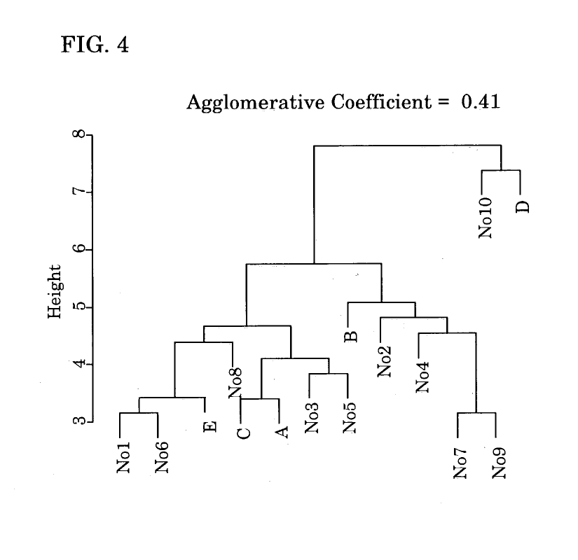

A total of fifteen samples including the samples of the five subjects and the samples of the ten patients mentioned above were subjected to agglomerative hierarchical clustering analysis using, as indices, changes with time of the expression levels of genes of CCR5, CXCR3, CCR4,. CCR3, CCR8, CXCR5, MIP-1α, IP-10, TARC, MDC, SDF-1, IL4, IL10, IL12A, IL12B, IL18, TGFA, TGFB1, TGFB2, and TGFB3 in addition to the genes shown in Table 1. The data used herein were derived from the blood drawn before treatment and three months after the initiation of the treatment. The results are shown in FIG. 4. The ten patients mentioned above have identification numbers of No. 1 to No. 10, respectively, and the new five subjects have identification symbols of A, B, C, D, and E, respectively. FIG. 4 shows that the subject D among the new five subjects was in a group very near to that of the patient No. 10, and the other four subjects were classified into another group. It is evaluated that the interferon β treatment will have sufficient efficacy on the patient D among the new five subjects, since that on the patient No. 10 exhibited sufficient efficacy, as described above.

In contrast, the results of MRT tests and clinical findings of the new five subjects show that only the subject D exhibited remarkable improvement in symptoms six months after the initiation of the interferon β treatment.

As is described above, the evaluation by means of gene expression very satisfactorily agrees with the results based on the MRI tests and clinical findings, demonstrating that the present invention is very effective.

The present invention has been accomplished based on the study on an evaluation method of the efficacy by determining a specific gene cluster in leukocytes derived from the peripheral blood of patients with MS by means of a simple and easy procedure such as DNA chips. The evaluation method of the present invention can easily and precisely evaluate the interferon β treatment.

While the present invention has been described with reference to what are presently considered to be the preferred embodiments, it is to be understood that the invention is not limited to the disclosed embodiments. On the contrary, the invention is intended to cover various modifications and equivalent arrangements included within the spirit and scope of the appended claims. The scope of the following claims is to be accorded the broadest interpretation so as to encompass all such modifications and equivalent structures and functions.

What is claimed is:

1. An evaluation method of an interferon β treatment, comprising the steps of:
labeling, with a fluorescent dye, a messenger RNA sample derived from peripheral blood leukocytes of a subject;
mixing and thereby hybridizing the fluorescence-labeled sample with probes corresponding to at least one interferon induced protein gene, at least one interferon regulation factor gene, and at least one chemokine gene;
detecting fluorescence to thereby determine the expression levels of the at least one interferon induced protein gene, the at least one interferon regulation factor gene, and the at least one chemokine gene;
referring to a database comprising data on correlation between the efficacy of an interferon β treatment and the expression levels of the at least one interferon induced protein gene, the at least one interferon regulation factor gene, and the at least one chemokine gene; and
evaluating the efficacy of the interferon β treatment on the subject based on the measured gene expression levels and the correlation data.

2. The evaluation method according to claim 1, further comprising using at least one gene having a symbol name selected from the group consisting of IFIT1, IFIT4, G1P3, and ISG15 as the at least one interferon induced protein gene, using at least one gene having a symbol name selected from the group consisting of IRF1, IRF2, IRF3, IRF4, IRF5, IRF6, and IRF7 as the at least one interferon regulation factor gene, and using at least one gene having a symbol name selected from the group consisting of SCYA2, SCYA22, SCYA5, SCYB14, CCR5, CXCR3, CCR4, CCR3, CCR8, CXCR5, MIP-1α, MTG, IP-10, TARC, MDC, and SDF-1 as the at least one chemokine gene.

3. The evaluation method according to claim 2, further comprising using probes corresponding to at least one interleukin gene having a symbol name selected from the group consisting of IL4, IL10, IL12A, IL12B, and IL18, and to at least one transforming growth factor gene having a symbol name selected from the group consisting of TGFA, TGFB1, TGFB2, and TGFB3;
wherein the database further comprises data on correlation between the efficacy of the interferon β treatment and the expression levels of the at least one interleukin gene and the at least one transforming growth factor gene.

4. An oligonucleotide array for evaluating an interferon β treatment, comprising:
a substrate, and
probes immobilized on the substrate, the probes corresponding to at least one interferon induced protein gene, at least one interferon regulation factor gene, and at least one chemokine gene, all of which vary in their gene expression levels with the interferon β treatment.

5. The oligonucleotide array according to claim 4, wherein the at least one interferon induced protein gene is at least one gene having a symbol name selected from the group consisting of IFIT1, IFIT4, G1P3, and ISG15, wherein the at least one interferon regulation factor gene is at least one gene having a symbol name selected from the group consisting of IRF1, IRF2, IRF3, IRF4, IRF5, IRF6, and IRF7, and wherein the at least one chemokine gene is at least one gene having a symbol name selected from the group consisting of SCYA2, SCYA22, SCYA5, SCYB14, CCR5, CXCR3, CCR4, CCR3, CCR8, CXCR5, MIP-1α, MIG, IP-10, TARC, MDC, and SDF-1.

6. The oligonucleotide array according to claim 5, further comprising probes immobilized on the substrate, the probes corresponding to at least one interleukin gene having a symbol name selected from the group consisting of IL4, IL10, IL12A, IL12B, and IL18, and to at least one transforming growth factor gene having a symbol name selected from the group consisting of TGFA, TGFB1, TGFB2, and TGFB3.
